(19) United States
(12) Patent Application Publication
(10) Pub. No.: US 2010/0061965 A1
(43) Pub. Date: Mar. 11, 2010

(54) RESPIRATORY SYNCYTIAL VIRUS RENDERS DENDRITIC CELLS TOLEROGENIC

(75) Inventors: JOHN E. CONNOLLY, DALLAS, TX (US); JACQUES F. BANCHEREAU, DALLAS, TX (US); MICHELLE A. GILL, PLANO, TX (US)

Correspondence Address: CHALKER FLORES, LLP, 2711 LBJ FRWY, Suite 1036, DALLAS, TX, 75234, US

(73) Assignee: BAYLOR RESEARCH INSTITUTE, DALLAS, TX (US)

(21) Appl. No.: 12/479,253

(22) Filed: Jun. 5, 2009

Related U.S. Application Data
(60) Provisional application No. 61/059,650, filed on Jun. 6, 2008.

(51) Int. Cl. A61K 45/00; C12N 5/0784; C12N 5/0783; A61P 37/06

(52) U.S. Cl. 424/93.7; 435/377; 435/325

(57) ABSTRACT

The present invention includes compositions, methods and systems for inducing immune tolerance using antigen presenting cells by infecting isolated antigen presenting cells with an effective amount of respiratory syncytial virus (RSV) or portions thereof sufficient to infect the antigen presenting cells and contacting CD4⁺, CD8⁺ or both CD4⁺ T cells and CD8⁺ T cells with the RSV-infected antigen presenting cells, wherein the CD4⁺, CD8⁺ or both CD4 and CD8+ T cells are rendered tolerogenic as measured in vitro by a mixed leukocyte reaction.

### CROSS-REFERENCE TO RELATED APPLICATIONS

This application claims priority to U.S. Provisional Application Ser. No. 61/059,650, filed Jun. 6, 2008, the contents of which is incorporated by reference herein in its entirety.

### STATEMENT OF FEDERALLY FUNDED RESEARCH

This invention was made with U.S. Government support under grants from the National Institutes of Health (U19 AIO57234, R21 AI 054990 and K08 AI059379-02). The government has certain rights in this invention.

### TECHNICAL FIELD OF THE INVENTION

The present invention relates in general to the field of immune cell tolerance, and more particularly, to compositions and methods for inducing immune suppression.

### INCORPORATION-BY-REFERENCE OF MATERIALS FILED ON COMPACT DISC

None.

### BACKGROUND OF THE INVENTION

Without limiting the scope of the invention, its background is described in connection with tolerogenicity.

U.S. Pat. No. 6,936,468 issued to Robbins, et al., teaches the use of tolerogenic dendritic cells for enhancing tolerogenicity in a host and methods for making the same. Briefly, the method relates to tolerogenic mammalian dendritic cells (DCs) and methods for the production of the tolerogenic DCs. In addition, a method is taught for enhancing tolerogenicity in a host comprising administering the tolerogenic mammalian DCs of the present invention to the host. The tolerogenic DCs includes a oligodeoxyribonucleotide (ODN) which has one or more NF-κB binding sites. The tolerogenic DCs of the present invention may further comprise a viral vector, and preferably an adenoviral vector, which does not affect the tolerogenicity of the tolerogenic DCs when present therein. Enhanced tolerogenicity in a host is useful for prolonging foreign graft survival and for treating inflammatory related diseases, such as autoimmune diseases.

U.S. Pat. No. 5,597,563 issued to Beschorner teaches a method induction of antigen-specific immune tolerance. The method for inducing antigen-specific immune tolerance by depletion of resident thymic antigen presenting cells (APCs) and re-population of thymus with new APCs containing the antigen for tolerance includes administering to a recipient animal a dendritic cell depleting amount of an immunosuppressive agent, for a time and under conditions sufficient for depletion of the dendritic cells in the recipient's thymic medulla, administering to the recipient animal a tolerogenic, amount of an intraspecies dendritic cell population in combination with the antigen, substantially contemporaneously with the immunosuppressive agent wherein the intraspecies dendritic cell population is enriched with intraspecies dendritic cells tolerogenic for the antigen and the administering is under conditions sufficient to repopulate the recipient's dendritic cell-depleted thymic medulla; and administering a thymic regeneration agent for a time and under conditions sufficient to induce recruitment of dendritic cells to the thymus, wherein the thymic regenerating agent is administered following the immunosuppressive agent and simultaneously or following administration of dendritic cells.

United States Patent Application No. 20060182726, filed by Thomas, et al., teaches immunomodulating compositions, processes for their production and uses therefore. The application discloses compositions and methods for antigen-specific suppression of immune responses, including primed immune responses. In particular, the invention discloses antigen-presenting cells, especially dendritic cells, whose level and or functional activity of CD40, or its equivalent, is impaired, abrogated or otherwise reduced, and their use for treating and/or preventing unwanted or deleterious immune responses including those that manifest in autoimmune disease, allergy and transplant rejection.

United States Patent Application No. 20040072348, issued to Leishman, teaches tolerogenic antigen-presenting cells. Dendritic cells can be prepared that cannot mature but that provide a first signal to T cells but cannot provide the co-stimulatory signal. T cells that are stimulated by the permanently immature dendritic cells therefore anergise, so the dendritic cells are tolerogenic rather than immunogenic. The cells are generally CD40⁻, CD80⁻ and CD86⁻, and remain so when stimulated by inflammatory mediators such as lipopolysaccharide. The cells can be prepared conveniently by the culturing adherent embryonic stem cells in the presence of GM-CSF.

Finally, United States Patent Application No. 20040043483, filed by Qian, teaches novel tolerogenic dendritic cells and therapeutic uses therefore. The application relates to tolerogenic dendritic cells (DCs) and methods for enriching for these cells in tissue preparations and using the cells for preventing or minimizing transplant rejection or for treating or preventing an autoimmune disease.

### SUMMARY OF THE INVENTION

The present invention includes compositions and method for inducing immune tolerance using antigen presenting cells. In one embodiment, the present invention includes anergic or tolerized immune cells and methods for making such cells by infecting isolated antigen presenting cells with an effective amount of respiratory syncytial virus (RSV) or portions thereof sufficient to infect the antigen presenting cells; and contacting CD4+, CD8+ or both CD4+ T cells and CD8+ T cells with the RSV-infected antigen presenting cells, wherein the CD4+, CD8+ or both CD4 and CD8+ T cells are rendered tolerogenic as measured in vitro by a mixed leukocyte reaction. In one aspect, the the RSV-infected antigen presenting cells are peripheral blood mononuclear cells, immature dendritic cells, mature dendritic cells or Langerhans cells. In another aspect, the RSV-infected antigen presenting cells are tolerogenic at a ratio of 1:1 to 1:100 tolerogenic antigen presenting cells to T cells. In another aspect, the RSV-infected cells are fixed prior to contacting with the T cells. The cells made using the method may be RSV-infected antigen presenting cells that are CD80high, CD86high, CD40high and CD83low. In another aspect, the RSV-infected antigen presenting cells are CD80high, CD86high, CD40high and CD83low, when compared to Flu infected antigen presenting cells. It has been found that the RSV-infected antigen presenting cells induce the proliferation of regulatory T-cells. The RSV-infected antigen presenting cells secrete IL-10 and have increased expression over untreated antigen presenting cells of SIGLEC-1, PDL-1, ILT-4, HLA-G, SLAM and LAIR. The RSV-infected antigen presenting cells may also have an increase in gene expression, when compared to untreated antigen presenting cells, of IL-10, LAIR2, SOCS2, PTPN2, ILT-6, AQP9, PTX3 and SLAMF1.

In another embodiment, a method for making tolerizing dendritic cells includes infecting dendritic cells with effective amount of respiratory syncytial virus to develop IL-10 dependent tolerogenic immune function, wherein respiratory syncytial virus increased the dendritic cells' ability to tolerize allogeneic CD4+ T-cells, cause suppressor T-cell proliferation, secrete IL-10 and express inhibitory molecules PDL-1, ILT-4 and HLA-G and wherein the infecting dendritic cells are CD80high, CD86high, CD40high and CD83low. In another aspect, the inhibition of dendritic cells' ability to activate allogeneic CD4+ T-cell requires cell-to-cell contact between dendritic cells.

In another embodiment, the present invention includes a method for suppressing antiviral immunity of dendritic cells in a subject by infecting isolated dendritic cells with effective amount of respiratory syncytial virus to develop IL-10 dependent tolerogenic immune function, wherein respiratory syncytial virus inhibit the dendritic cells' ability to activate allogeneic CD4+ T-cells, induce naïve T-cell regulatory response, secrete IL-10 and express inhibitory molecules PDL-1, IKT-4, and HLA-G when reintroduced into a patient. In one aspect, the inhibition of dendritic cells' ability to activate allogeneic CD4+ T-cell requires cell-to-cell contact between dendritic cells.

Another embodiment of the present invention is a tolerogenic dendritic cell comprising an isolated dendritic cell that is CD80high, CD86high, CD40high and CD83low. The tolerogenic dendritic cell made by the method of infecting peripheral blood mononuclear cells with an effective amount of a respiratory syncytial virus or portions thereof sufficient to rendered CD4+, CD8+ or both CD4+ T cells and CD8+ T cells tolerogenic as measured in vitro by a mixed leukocyte reaction and wherein the dendritic cells that is CD80high, CD86high, CD40high and CD83low.

Another embodiment of the present invention is a method of promoting tolerogenic T cell-mediated immune responses by contacting the T cells with a dendritic cell that has been infected with an amount of a RSV or portion thereof sufficient to trigger the surface expression of at least one of CD80high, CD86high, CD40high and CD83low. Another embodiment is a method of inducing anergic T helper cells that includes incubating isolated antigen presenting cells (APC) with an amount of RSV sufficient to infect the antigen presenting cell and trigger the surface expression of at least one of the following cell surface markers CD80high, CD86high, CD40high and CD83low; and contacting the RSV-infected antigen presenting cells with T cells under conditions that tolerize the T cells as measured in vitro in a mixed lymphocyte reaction.

Another embodiment of the present invention is a method of producing an isolated tolerogenic dendritic cell by incubating the isolated dendritic cell with an amount of respiratory syncytial virus sufficient to infect the dendritic cell under conditions that trigger the cell surface expression the following cell surface CD80high, CD86high, CD40high and CD83low. The present invention also includes a kit for enhancing tolerogenicity in a mammalian host comprising isolated tolerogenic dendritic cells previously infected with RSV and having the following cell surface CD80high, CD86high, CD40high and CD83low.

Yet another embodiment of the present invention includes a method of generating a tolerogenic antigen presenting cell (APC) by infecting the APC with an amount of respiratory syncytial virus sufficient to infect the dendritic cell; and causing the following cell surface marker expression CD80high, CD86high, CD40high and CD83low thereby generating a tolerogenic antigen presenting cell (APC). A method may also be used to treat an autoimmune disease in a mammalian subject, comprising administering to the mammalian subject tolerogenic antigen presenting cell (APC), wherein the tolerogenic dendritic cells previously infected with RSV and having the following cell surface CD80high, CD86high, CD40high and CD83low, and the cells are administered in an amount effective to reduce or eliminate the autoimmune disease or to prevent its occurrence or recurrence. Non-limiting examples of autoimmune diseases that may be treated using the present invention includes insulin-dependent diabetes mellitus, multiple sclerosis, autoimmune encephalomyelitis, rheumatoid arthritis, autoimmune arthritis, myasthenia gravis, thyroiditis, uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, psoriasis sympathetic ophthalmia, phacogenic uveitis, autoimmune haemolytic anaemia, idiopathic leucopenia, primary biliary cirrhosis, active chronic hepatitis, cryptogenic cirrhosis, ulcerative colitis, Sjogren's syndrome, scleroderma, Wegener's granulomatosis, poly/dermatomyositis, discoid lupus erythematosus or systemic lupus erythematosus.

In another embodiment, the present invention includes a method for modulating the immune response to an antigen, by administering to a patient in need of such treatment an isolated tolerizing antigen-presenting cell for a time and under conditions sufficient to modulate the immune response, wherein the antigen-specific antigen-presenting cell is produced by contacting the antigen-presenting cell with RSV for a time and under conditions sufficient for the antigen-presenting cell to become a tolerizing to T cells, wherein the tolerizing antigen-presenting cell is characterized by expressing the following cell surface markers CD80high, CD86high, CD40high and CD83low, and wherein the tolerizing antigen presenting cell is tolerogenic at a ratio of 1:5 to 1:100 tolerizing antigen presenting cells to T cells.

### BRIEF DESCRIPTION OF THE DRAWINGS

For a more complete understanding of the features and advantages of the present invention, reference is now made to the detailed description of the invention along with the accompanying figures and in which:

FIG. 1A shows PBMCs isolated from pediatric patients with acute RSV infection and healthy adult donors. The proliferation of healthy donor CFSE labeled CD4+ T-cells were assessed by flow cytometry following 6 day coculture with irradiated PBMCs from RSV infected or health individuals.

FIG. 1B shows mDCs isolated by direct sorting of CD11c+ HLA-DR+cells from nasal mucosal washes of acutely infected infants. Cells were cultured with CFSE labeled CD4+ T-cells and proliferation was assessed by flow cytometry.

FIG. 2A shows Blood mDCs isolated as described and exposed to either Flu or RSV (MOI=1.0) or unexposed for 18 hours and washed extensively. Their ability to promote CD4+ T-cell proliferation was then assessed.

FIG. 2B shows 2×10̂4 cells mDCs were exposed to either Flu or RSV (MOI=1), or UV-irradiated RSV (MOI=1) for 24 hours at 37C. 1.5×10̂5 CSFE labeled T cells were incubated with 2.5×10̂3 Flu, RSV, or UV-RSV treated mDCs for 6 days. Cells proliferation assessed by CFSE dye dilution.

FIG. 2C shows DCs differentiated in the presence of GM-CSF and IL-4 or GM-CSF and IL-10 or GM-CSF and Vitamin D3 for 6 days or combinations as indicated. At day 6, drug treated DCs incubated for 2 days in presence LPS. At day 8 the ability of these DCs to promoted CD4+ T-cell proliferation was assessed by thymidine incorporation relative to RSV DCs.

FIG. 2D shows blood mDCs isolated as described in FIG. 1. CD4+ T-cells were purified by cell sorting and then CSFE labeled. 1.5×10⁵ cells were exposed to 1 MOI of either Flu, RSV or control conditions. Cells were then incubated for 6 days at 37C in the presence of anti-CD3, anti-CD28 microbeads. Cells were then stained for CD4 expression and proliferation assessed by CFSE dye dilution.

FIG. 2E shows blood mDCs (green triangles) and plasmacytoid DCs (pDCs) (blue X) isolated using the method outlined in FIG. 1. Cells were inoculated with RSV (MOI=1) in 96-well plates. Hela cells (red X) served as a positive control for viral replication. RSV cultured in the absence of cells (yellow circles) served as a negative control. Infectious viral particle production was assessed every 24 hours, for 7 days. Freshly thawed vials of RSV (dark blue diamonds) were used to confirm tissue culture infectious dose (TCID50) calculation.

FIG. 2F shows blood mDCs isolated using the method outlined in FIG. 1, DCs derived from GM-CSF IL4 cultured monocytes were isolated on day 6 (GM/4 DC). Cells were cultured with either influenza virus (MOI=1) or RSV (MOI=1) in 96-well plates. After 24 hours, cell viability was assessed by trypan blue staining Data represent the mean and standard deviation of 4 independent studies

FIG. 3A (panel 1) shows mDCs purified and exposed for 18 hours to either no virus, RSV (blue) or Flu virus (red). Eighteen hours later, allogeneic CFSE-labeled CD4+ T cells were cocultured with unexposed DCs plus increasing numbers of RSV-exposed or Flu exposed DCs. After 6 days of coculture CD4+ T-cell proliferation was assessed by flow cytometry. FIG. 3A (panel 2) shows the results from 5 independent studies with donor matched mDCs (p=0.03 paired t-test).

FIG. 3B shows mDCs prepared as described in FIG. 3A, with increasing numbers of unexposed DCs (circles) or RSV exposed (triangles) DCs titrated into the mDC/CD4+ T-cell coculture either directly (blue) or on across a 0.3 uM trans well. CD4+ T-cell proliferation was assessed as described above.

FIG. 3C shows blood mDCs exposed for 18 hours to either no virus (control), RSV or Flu virus. Exposed dendritic cells were then fixed for 30 min at room temp using CytoChex fixation reagent (BD) and washed 3 times with ice cold PBS. Flowing viral exposure and fixation, cells were used in an in trans allo inhibition assay. Allogeneic CFSE-labeled CD4+ T-cells were cocultured with unexposed DCs plus increasing numbers of RSV-exposed or Flu exposed DCs, either fixed or unfixed. After 6 days of coculture CD4+ T-cell proliferation was assessed by flow cytometry.

FIG. 3D shows DCs prepared as described in FIG. 1. Increasing numbers of virally or pharmacologically manipulated DCs were added to mDC/CD4+ T-cell cocultures. CD4+ T-cell proliferation was assessed as described above.

FIG. 3E shows blood mDCs were cultured for 24 hours with flu or RSV (MOI=1) or no virus (control), and harvested. Control mDCs (no viral exposure), were incubated in the presence of increasing concentrations of either flu or RSV-exposed DCs with or without 5.0 ug/ml anti-F protein antibody, and co-cultured with CFSE-labeled allogeneic CD4+ T cells at a constant concentration of 1,250 control mDCs per 100,000 CD4+ T cells. After 6 days, cells were stained for CD4 expression and proliferation was assessed by CFSE dye dilution.

FIG. 4A shows mDCs isolated using the method of the present invention and cultured with either Flu or RSV (MOI=1). After 24 hours, cells were stained for CD40, CD83, CD86, and CD80 and analyzed by flow cytometry. Pink histograms represent unexposed mDCs stained for the same markers. Green histograms represent mDCs cultured with RSV and blue histograms represent mDCs cultured with influenza.

FIG. 4B shows the results of mDCs (3 donors) exposed to either Flu or RSV (MOI=1.0) or unexposed for 16 hours, after which RNA was extracted, labeled and hybridized to the U133 2 plus chip (Affymetrix). Differential analysis was performed using Gene Spring 6.2 software package (Silicon Genetics). Expression pattern of 15 probes associated with tolerogenic DCs from mDCs exposed to either flu or RSV or unexposed.

FIG. 4C shows the expression of ITIM receptors and ligands as assessed by flow cytometry following an 18 hour exposure with either Flu or RSV.

FIG. 4D shows the expression of IFN lambda and IFN alpha family members in mDCs following 18 hours of Flu or RSV (MOI=1), 3 donors relative to unexposed mDCs.

FIG. 4E shows the results from blood mDCs isolated as described in FIG. 1. DCs were either, untreated (control), treated with Flu (MOI=1), with RSV (MOI=1), or with IFN-alpha (500 pg/ml), and IFN-lambda and IL-29 (1 or 5 ng/ml) for 24 hrs at 37C. Allogeneic CFSE-labeled CD4+ T-cells were then cocultured with DCs of each treatment for 6 days at 37C. In the case of cytokine treated DCs, IFN-lambda, IFN-alpha and IL-29 concentrations were maintained throughout the T-cell coculture. After 6 days of coculture CD4+ T-cell proliferation was assessed by flow cytometry (upper panel). The bioactivity of recombinant IFN-lambda and IL-29 were assessed by monitoring STAT-1 phosphorylation in exposed GM-CSF/IL-4 monocyte derived DCs. Monocyte derived DCs were exposed to 5 ng/ml IFN-lambda and IL-29 for 0, 10, 30 and 60 minutes respectively. The degree of STAT-1 phosphorylation (P-STAT1) was then assessed in whole cell lysates by western blot relative to total STAT-1 protein. Monocyte derived DCs treated for 60 minutes with 500 pg/ml IFN-alpha served as a positive control for STAT-1 phosphorylation (lower panel).

FIG. 5A shows the results from mDCs exposed to 1 MOI of either Flu or RSV for 18 hours. Cell Culture Supernatants were analyzed for the expression of IL-10 Luminex Multiplex Analysis. Data represents the mean and SD of 11 independent studies.

FIG. 5B shows the results from mDCs incubated with either isotype control (panels 1 and 3) or blocking antibodies to IL-10 and IL-10 receptor (panels 2 and 4), either 30 min prior to viral exposure (panel 2) or following 18 hours of RSV exposure (panel 4). The ability of these cells to induce CD4+ T-cell allo proliferation was then assessed (n=4).

CFSE labeled allogeneic CD4+ T-cell were cultured with either unexposed, flu or RSV exposed mDCs.

FIG. 6A shows the first and second generation CD4+ T-cells populations (CFSE high) from each condition, were isolated by cell sorting following 5 days of DC coculture. 1,500 CD4+ T-cells sorted from unexposed, flu or RSV exposed mDCs cocultures were thus added to MLR consisting of 1,250 unexposed mDCs and 500,000 labeled CD4+ T-cells. The ability of unexposed DCs to induce CD4+ T-cell allo proliferation was then assessed.

FIG. 6B and FIG. 6C shows the data from three independent studies are shown.

FIG. 6D. Schematic of the study process.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Respiratory Syncytial Virus (RSV) infection is the primary cause of hospitalization in the first year of life. Here we show that during the course of natural infection in infants, RSV blocks the antigen presenting function of dendritic cells (DCs). RSV exposed human DCs are incapable of activating naive CD4+ T-cells, they secrete IL-10 and express inhibitory molecules PDL-1, ILT-4, and HLA-G. RSV exposed DCs inhibit allogenic T-cell proliferation in mixed leukocyte reactions by a cell contact dependent mechanism. Furthermore, naive T-cells cocultured with RSV exposed DCs acquire regulatory T-cell function. It was found that RSV suppresses antiviral immunity by skewing DC maturation toward a tolerogenic phenotype and function.

Respiratory Syncytial Virus (RSV), a single-stranded RNA paramyxovirus, is the leading respiratory pathogen in infants and young children worldwide. RSV infection leads to incomplete immunity as children can get re-infected with the same strain of virus¹ and immunocompetent adults experience recurrent RSV infections²⁻⁴. The acute and long-term morbidity associated with RSV, makes an effective vaccine highly desirable. Unfortunately, early attempts at vaccine development led instead to sensitization to RSV suggesting unusual presentation of RSV to the adaptive immune system⁵⁻⁷.

Dendritic cells (DCs) are the primary antigen presenting cells (APCs) that guide the development and polarization of an adaptive immune response⁸ These cells are also a major target of viral immune evasion mechanisms^{9,10}. DCs have the unique ability to induce primary immune responses and control immune tolerance through the induction of both T-cell anergy and the generation of regulatory T-cells¹¹. Although initially theorized as the sole purview of immature DCs¹², recent work indicates that partially or even full mature DCs may play a central role in inducing immune tolerance in vivo¹³⁻¹⁸. The limited ability of immunocompetent individuals to mount protective immune responses against RSV, led us to investigate the status of APCs during acute viral infection and to analyze the response of DCs to RSV infection^{2,4}.

Mixed Leukocyte Reaction. PBMCs were isolated by density centrifugation from pediatric patients with acute RSV infection and healthy adult donors. PBMCs from the healthy “responder” were labeled with CFSE and cultured for 6 days at the concentration of 500 k per ml with varying concentrations of “stimulator” irradiated PBMCs from either RSV patients or healthy donors. The capacity of PBMCs from RSV patients versus non-infected donors to stimulate CD4+ T cell proliferation in responder PBMC cultures was measured at the following stimulator:responder PBMC ratios: 0:500 k, 125:500 k, 250:500 k, and 500:500 k. The proliferation of CD4+ T-cells within the healthy responder PBMCs were assessed by flow cytometry (as CFSE dye dilution) following 6 day coculture with irradiated PBMCs from RSV infected or healthy individuals.

Respiratory tract mDCs. Nasal wash samples were obtained by nasopharyngeal suctioning from hospitalized children with acute RSV or influenza infection. Cells from these samples were labeled with the LINEAGE-FITC (a cocktail of FITC-conjugated antibodies including anti-CD3, CD14, CD16, CD19, CD20, and CD56), CD123-PE, HLA-DR-PerCP, and CD11c-APC (BD Biosciences, San Jose, Calif.). mDCs were then isolated by direct sorting on a FACS ARIA as LINEAGE-negative, HLA-DR+, CD11c+ cells.

Blood mDCs. Leukocyte-enriched blood samples were obtained from a local blood bank. PBMCs were obtained using a Ficoll gradient (density centrifugation). PBMCs were then incubated with magnetic microbeads conjugated to anti-CD3, anti-CD14, anti-CD16, anti-CD19, and anti-CD56 and then passed over a magnetic column. The negative fraction was collected and stained for LINEAGE-FITC, CD123-PE, HLA-DR-PerCP, and CD11c-APC. The stained cells were then sorted on a FACS ARIA cell sorter. mDCs were defined as LINEAGEneg, HLA-DR+, CD11c+ cells. Purity of the isolated mDCs averaged 97%.

Cell Staining for Flow Cytometric Analysis. PBMCs or purified mDCs were incubated with 5 microliters of flouorochrome-conjugated anti-human antibodies for 30 minutes at 4 degrees C., rinsed with PBS, centrifuged at 1200 rpm for 5 minutes, and resuspended in 1% paraformaldehyde. Samples were then acquired on a FACSCalibur or FACS ARIA and analyzed with either Cellquest software (BD Biosciences, San Jose, Calif.) or FloJo software (Tree Star Inc., Ashland, Oreg.). The following fluorochrome-conjugated anti-human antibodies were used: CD83-FITC, HLA-DR-Per-CP, CD86-Alexa-405, CD80-FITC, and CD40-PE (for purified mDC studies) and CD8PE, CD3-PerCP, and CD4-APC (for PBMC studies).

CFSE Staining Cells were incubated at a concentration of 1-5 million cells per 0.5 ml in 1.25 micromolar CarboxyFluoroscein Succinimidyl Ester (CFSE) for 10 minutes, centrifuged at 1200 rpm for 5 minutes, and washed with 1 ml of a solution of RPMI 1640/10% human AB serum at 4 degrees. Centrifugation and washing steps were repeated twice followed by resuspension of the cells with 1640 RPMI/10% human AB serum. Cell proliferation was assessed by monitoring dye dilution of CFSE on stained CD4+ T-cells. In some studies, CFSE was used to identify T-cell populations which had divided once or twice. These populations were subsequently sorted and used in T-cell proliferation assays described in the text.

Quantitation of virus replication. RSV replication was assessed by tissue culture infectious dose (TCID50) calculation. TCID50 is defined as the dilution of assay sample at which 50% of a susceptible Hela cell culture inoculated becomes infected. Briefly, TCID50 value: −m=log10 starting dilution−[p-0.5]×d. The equation is defined where m is the log10 TCID50 (per unit volume inoculated per replicate culture), d is the log10 dilution factor, and p is the proportion of wells positive for viral infection.

In vitro viral exposure of mDCs. Purified blood mDCs were cultured for 18-24 hours at a concentration of 25,000 mDCs per 200 microliters in 96-well plates with influenza A virus (A/PR/8/34 H1N1 from Charles Rivers Laboratories, Wilmington, Mass.) or RSV A2 (generated on HeLA cells and purified via sucrose gradient) at a multiplicity of infection (MOI) of 1.

Preparation of tolerogenic DCs. PBMC were purified from human peripheral blood by Ficoll-Hypaque centrifugation. Monocytes were purified by adherence and differentiated into moDC after 6 days in the presence of GM-CSF and IL-4 (DC GM+IL-4) or GM-CSF and IL-10 (100 ng/ml, R&D) (DC GM+IL-10) or GM-CSF and vitamin D3 (100 nM, Calbiochem)) (DC GM+Vit D3). At day 6, DC are washed and recultured for 2 days in the presence of GM-CSF or GM-CSF and Dexamethasone (10 nM, Sigma-Aldrich) or GM-CSF and vitamin D3. DCs were washed twice and 2500 DC were cultured with 10⁵ allogeneic T lymphocytes in 96-well U-bottom in 5% AB medium for 5 days (triplicate). 1 uCi [³H]-thymidine was added for the last 18 h of culture. Plates were harvested on a Tomtec Harvester 96 and proliferation detected on a Wallac microbeta trilux-u-scintillant counter (PerkinElmer, Wellesly, Mass., USA).

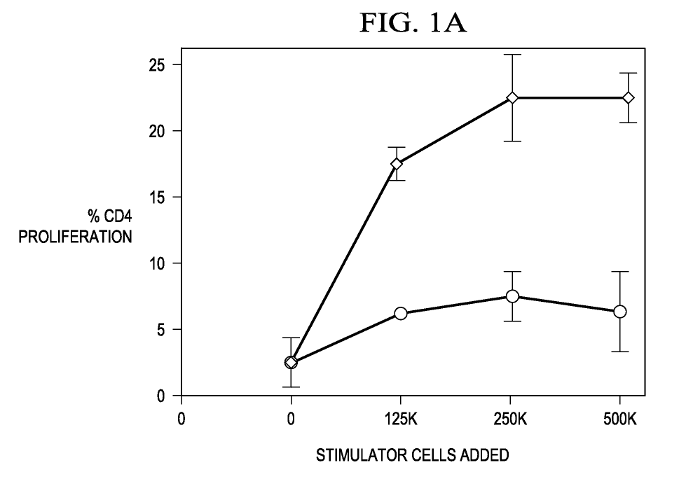

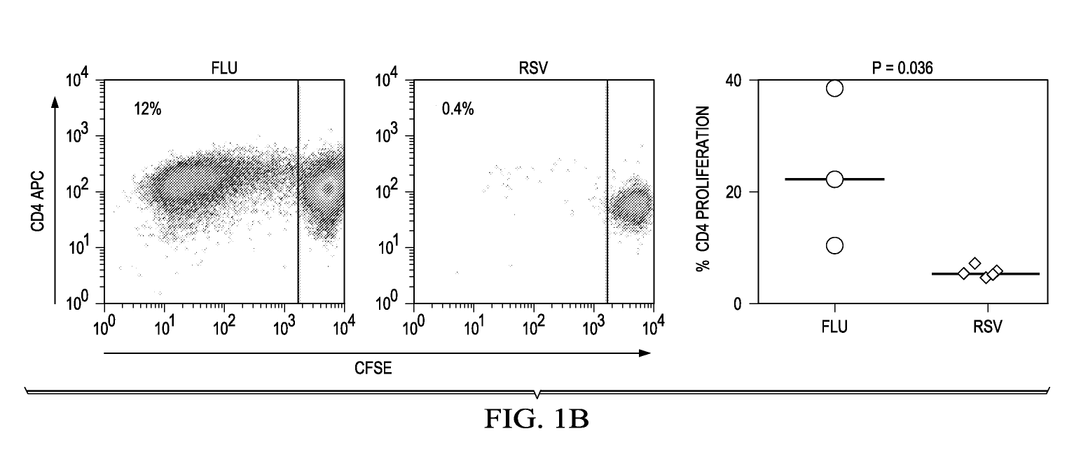

APCs from RSV infected patients do not activate allogeneic T-cells. As a measure of antigen presenting capacity, peripheral blood mononuclear cells (PBMCs) from patients acutely infected with RSV were tested for their ability to promote the proliferation of allogeneic CD4+ T-cells (mixed leukocyte reaction or MLR) by assessing the dilution of the CFSE dye. As shown in FIG. 1a, the PBMCs from a patient suffering from acute RSV infection (red line) failed to promote the proliferation of CD4+ T-cells from two healthy individuals. These CD4+ T-cells were able to proliferate when exposed to PBMCs from healthy individuals (green and blue lines). Since RSV replication occurs primarily in the upper respiratory track, we isolated DCs from the nasal mucosa of acutely infected RSV patients. Purified DCs were unable to promote allogeneic CD4+ T-cell proliferation (FIG. 1b center panel)¹⁹. In contrast, mDCs (CD11c+ HLA-DR high) isolated from influenza patients' mucosa were powerful stimulators of allogeneic T-cells. (FIG. 1b, left panel) This was observed with cells isolated from 6 patients infected with RSV and 3 patients infected with influenza (p<0.05) (FIG. 1b right panel). The mDCs isolated from one of these patients one month after resolution of RSV infection, were able to induce alloreactivity suggesting that the blocking effect of RSV on APC function is transient (data not shown) Thus antigen presenting cells, including DCs, from patients suffering from acute RSV infection do not present alloantigens efficiently.

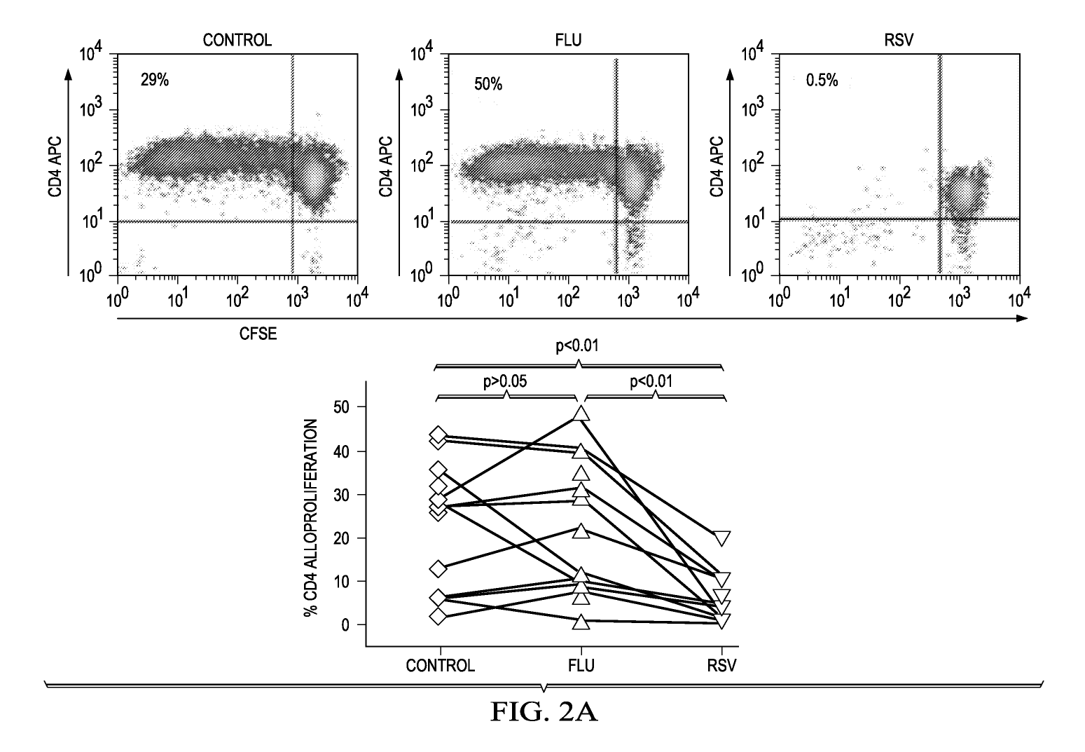

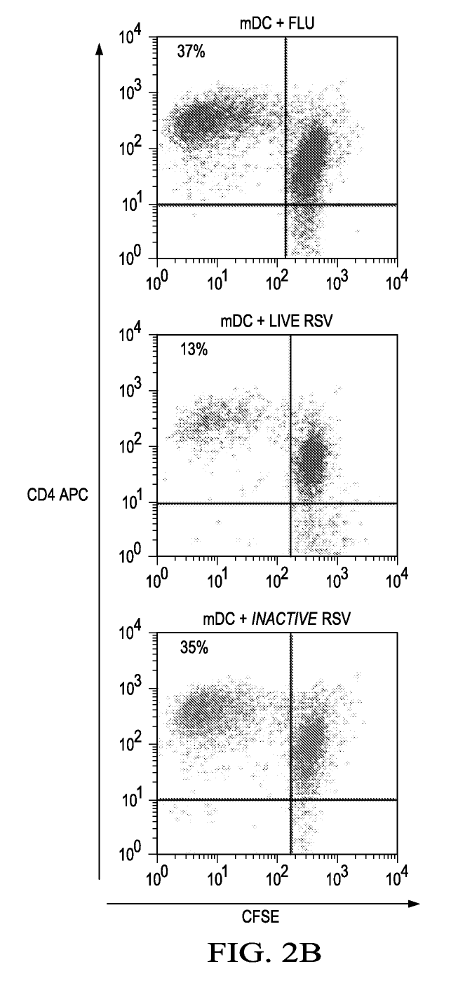

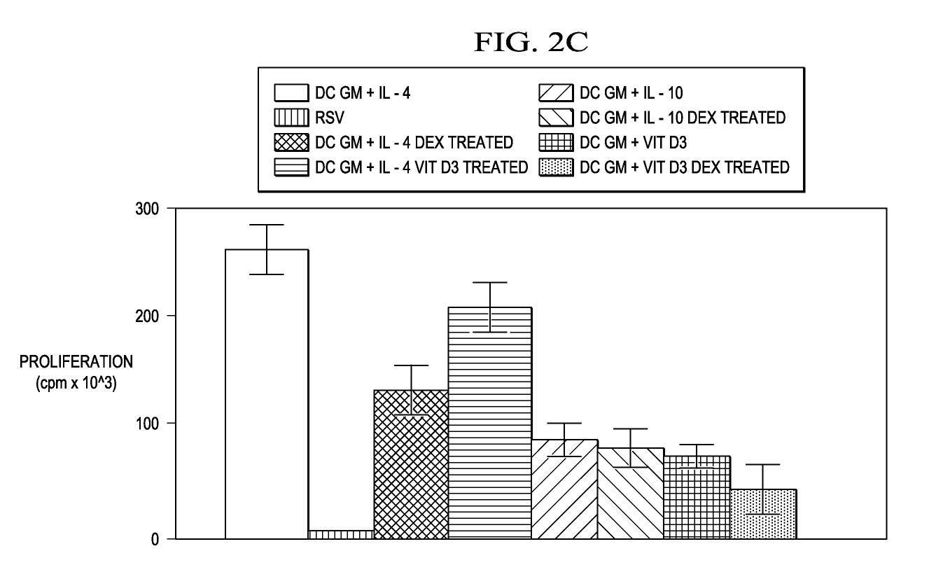

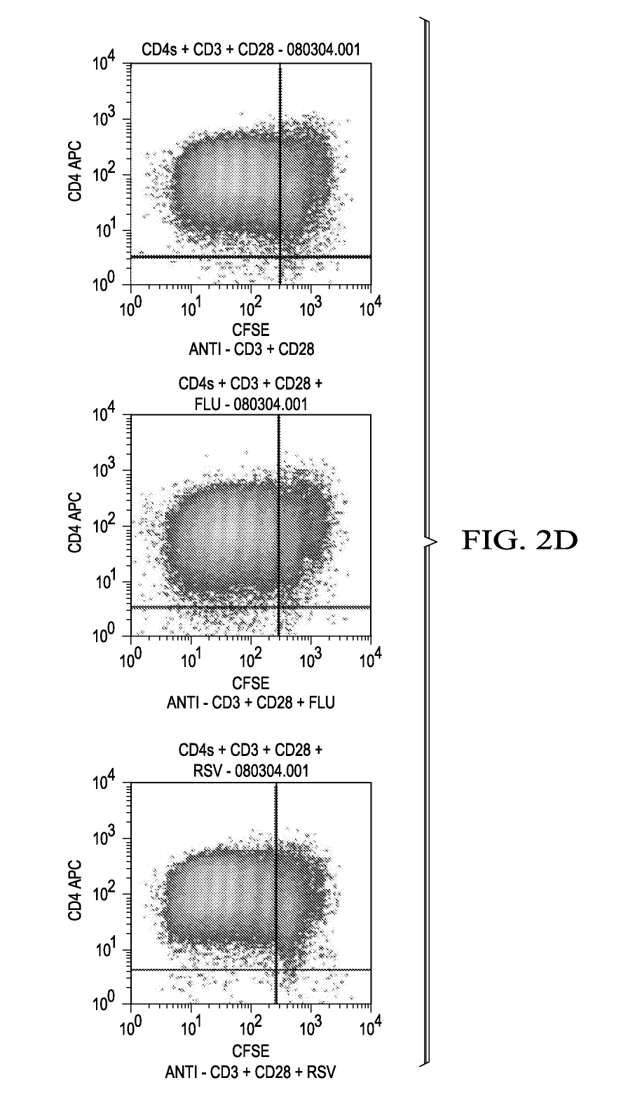

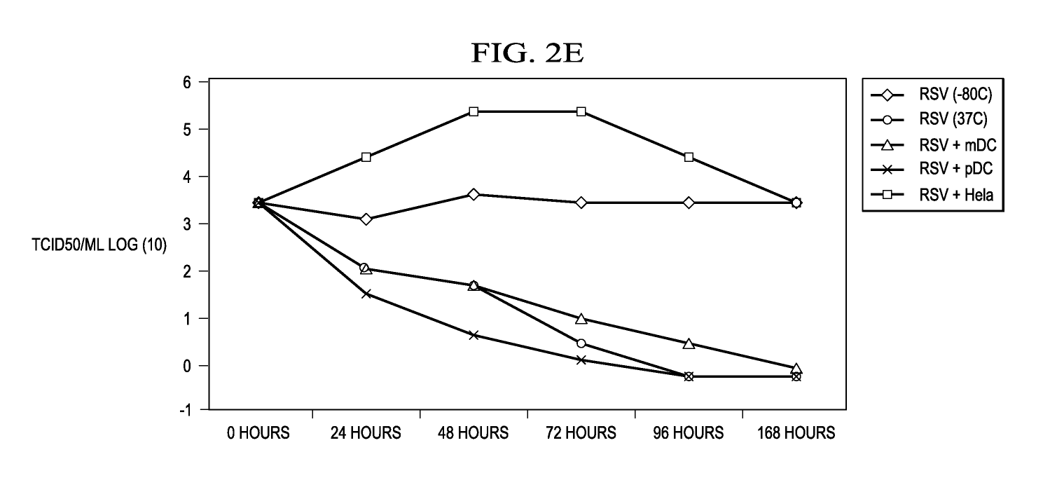

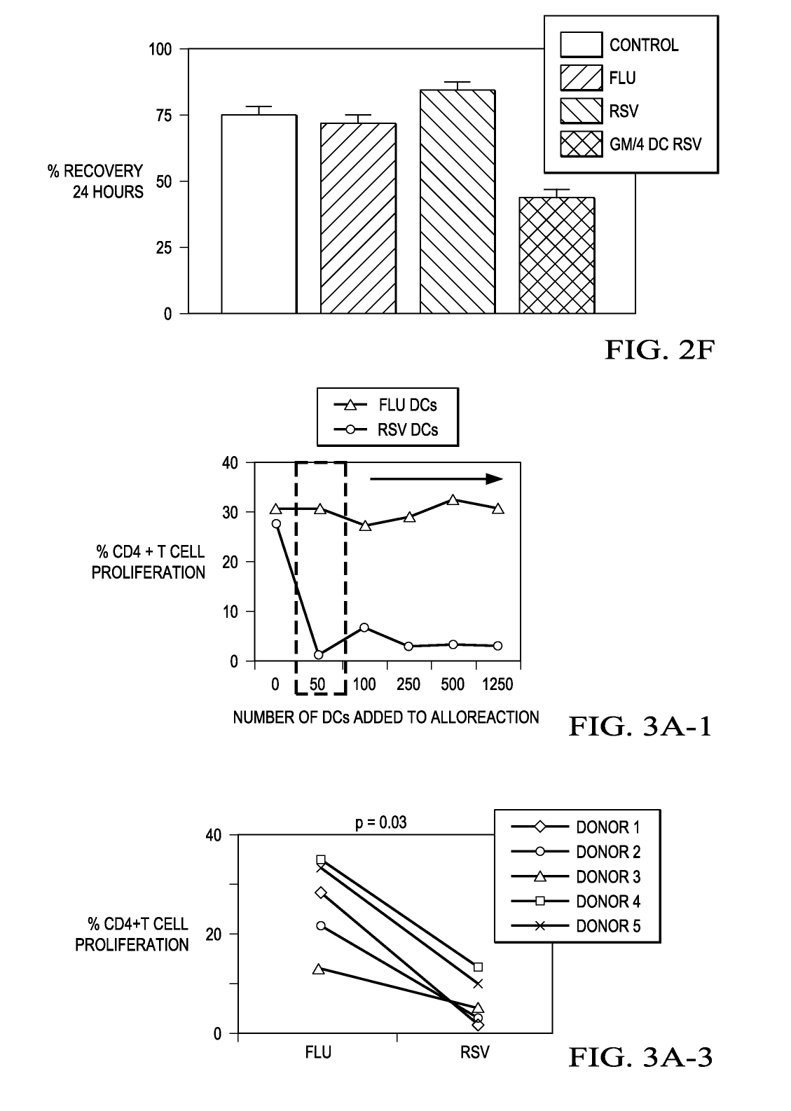

Blood mDCs exposed to RSV fail to induce an alloreaction. A series of in vitro studies were conducted to understand the mechanism by which RSV alters the antigen presenting capacity of DCs. Human mDCs isolated from peripheral blood by cell sorting (CD11c (+) HLA-DR (+) LIN), were exposed for 18 hours to either influenza (Flu) or respiratory syncytial virus (RSV). RSV exposed DCs (RSV-DCs) were unable to promote the proliferation of CSFE labeled allogeneic CD4+ T-cells while Flu exposed DCs were more efficient than unexposed DCs (FIG. 2A). The lack of proliferation was not due to RSV induced T cell death as virally exposed CD4+ T-cells proliferated in response to anti-CD3/28 (FIG. 2D). UV irradiation prior to mDC exposure inactivated the virus in terms of blocking allogeneic T cell stimulation, indicating a requirement for viral replication or nonstructural protein synthesis (FIG. 2B). An analysis of infectious particle production indicated that RSV does not replicate in primary human DCs (FIG. 2E). The inhibitory effect of RSV was not due to cell death as the viability of primary mDCs was equivalent among exposed and untreated cells (FIG. 2F). Conflicting reports exist as to the effect of RSV on GM-CSF/IL-4 monocyte derived DCs produced in vitro^{20,21}. Unlike primary human mDCs, a large fraction of monocyte derived DCs exposed to RSV die within 24 hours (FIG. 2F).

Others have reported that DCs with regulatory function can be produced in vitro by pharmacologic manipulation during their differentiation from monocyte precursors in the presence of GMCSF and IL-4. To compare the regulatory activity of RSV exposed DCs and monocyte derived DCs we produced the later in the presence of GM-CSF and either, dexamethasone^{22,23}, IL-10^{24 1}alpha,25-Dihydroxyvitamin-D(3)(VitD3)²⁵ or combinations there of and used them to stimulate allogeneic CD4+ T-cells. Each of these cell preparations demonstrated phenotypic markers of DC differentiation including CD11c, MHC Class-II and mannose receptor (CD206) and low levels of CD14, except for GM-CSF IL-10 cultured DCs which were CD14 high and DC-SIGN positive (data not shown). These pharmacologically manipulated DCs were less efficient at inducing MLR when compared to DCs generated with GM-CSF and IL-4. However in all cases these DCs induced significantly higher MLR when compared to RSV-DCs. (FIG. 2C).

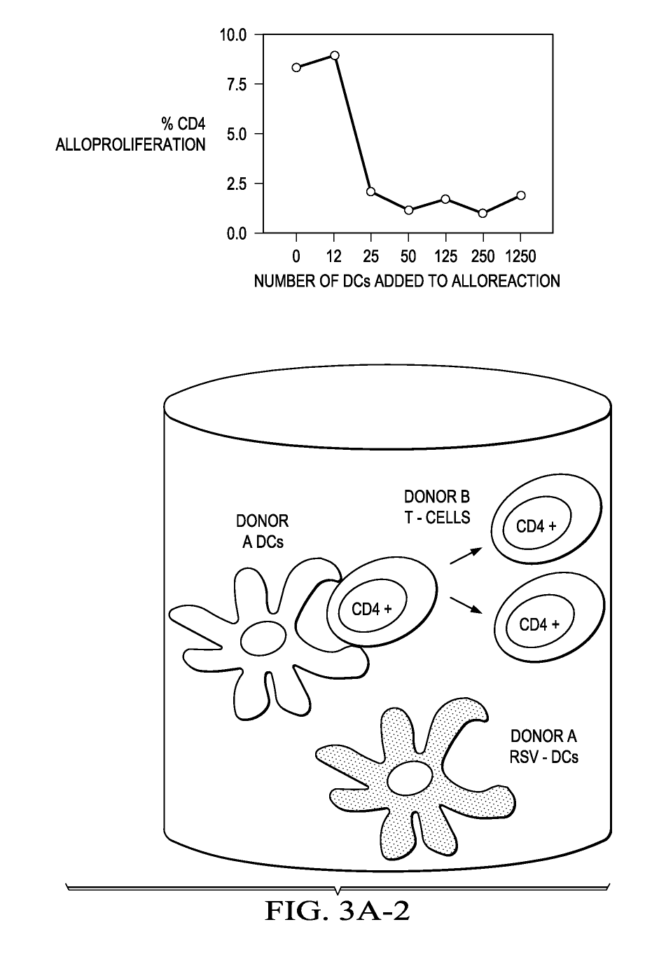

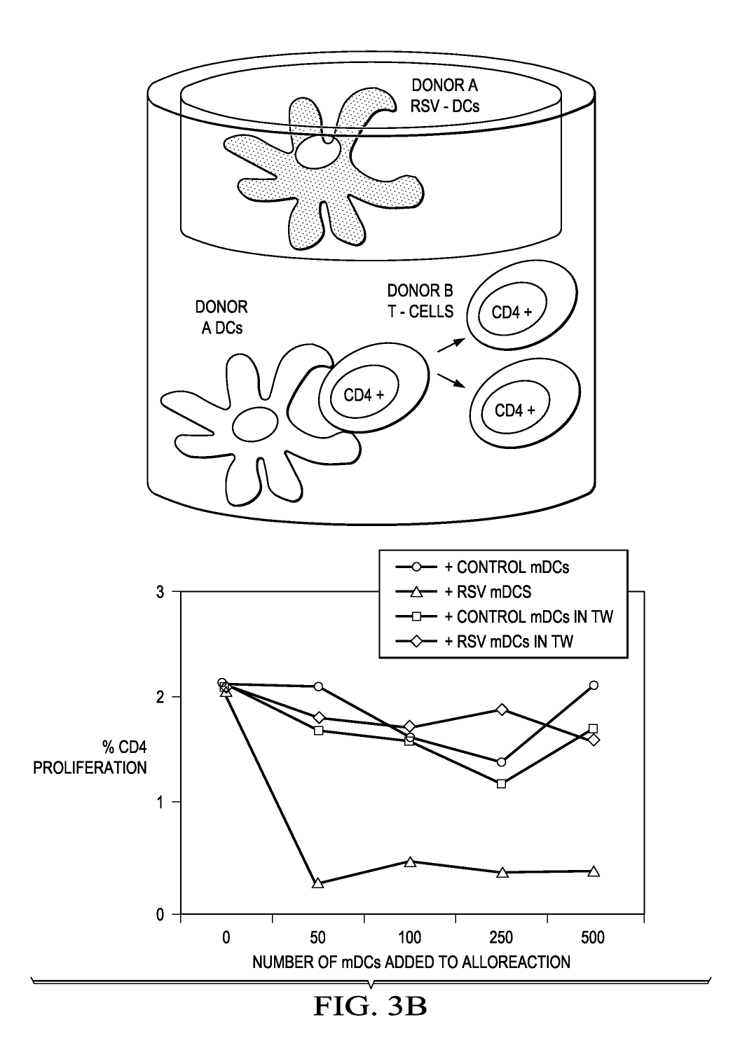

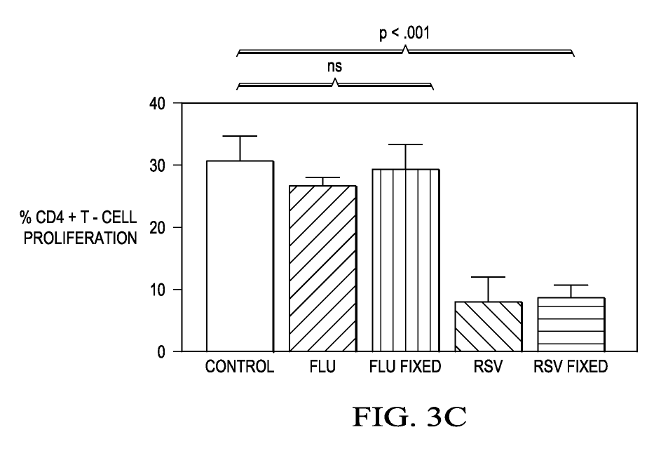

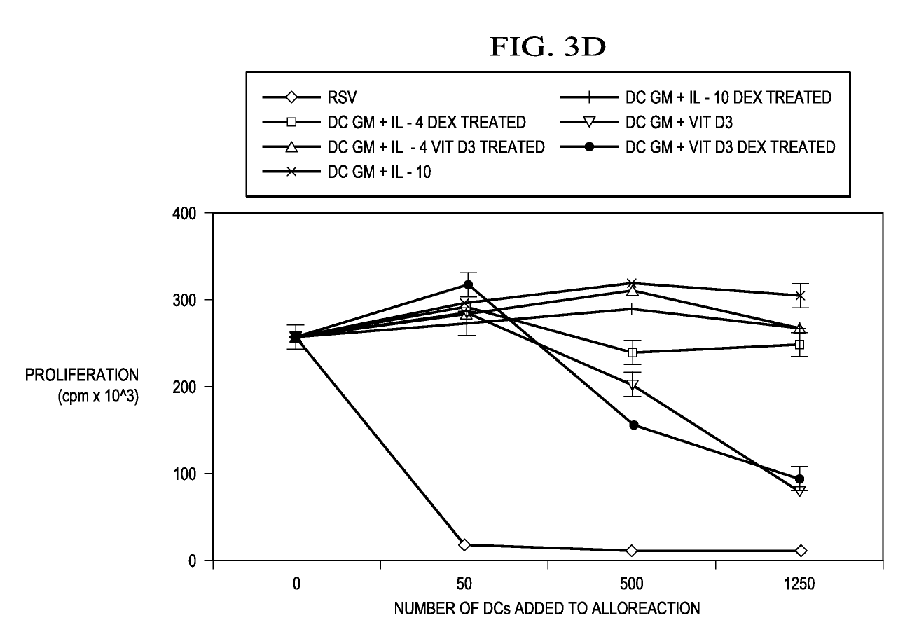

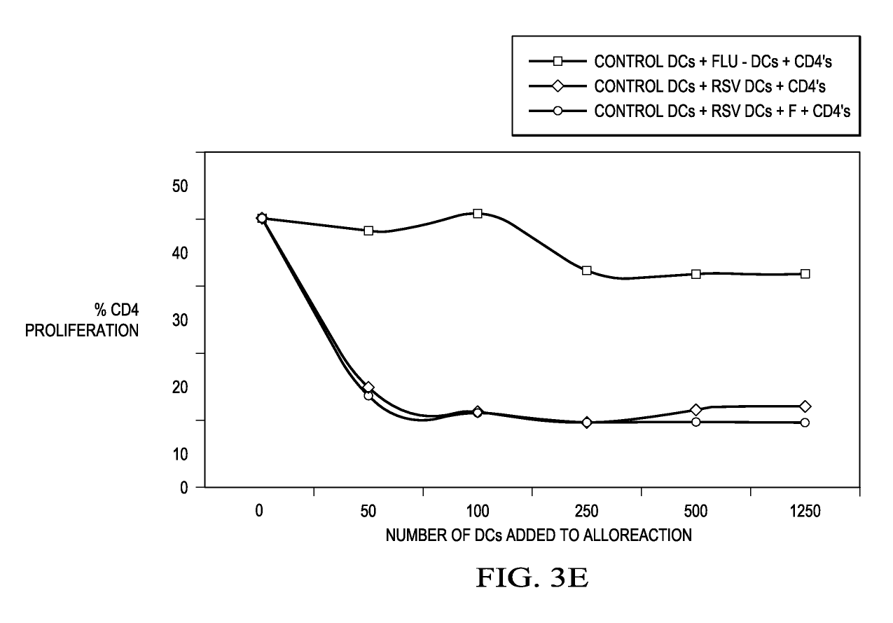

RSV DCs are potent suppressors of MLR. The inability of RSV DCs to stimulate allogeneic T cells led us to consider that RSV exposed mDCs might inhibit unexposed mDCs from promoting T-cell alloproliferation. An increasing number of either RSV or flu exposed mDCs from donor A were thus added to MLR consisting of 1,250 unexposed mDCs donor A and 100,000 labeled CD4+ T-cells from donor B. As shown in FIG. 3A panel 1 and 2 (blue line), as little as 25-50 RSV exposed mDCs inhibited unexposed mDC induced alloreaction by more than 85% (panel3, n=5 p<0.05) whereas Flu exposed DCs showed no effect (FIG. 3a panel 1, red line). Interestingly RSV infected DCs could block an MLR between DCs and T-cells from unrelated donors (data not shown). This inhibition was dependent on virally exposed mDCs, and was not due to carry over of RSV in the culture system, since the addition of a blocking antibody to the RSV fusion protein (F), failed to prevent the inhibitory capacity of RSV-DCs (FIG. 3E). This inhibition of MLR induced by RSV-DCs required direct cell to cell contact as RSV-DCs added to the top chamber of a 0.3 um transwell, did not inhibit the alloproliferative response in the lower well (FIG. 3B, blue line vs. green line). Furthermore RSV-DCs retained their suppressive capacity after paraformaldehyde fixation (FIG. 3C). Of the pharmacologically generated tolerogenic DCs, only VitD3 and VitD3 dexamethasone differentiated DCs were able to inhibit allo reactions in trans, yet their inhibitory capacity was far less than that of RSV-DCs (FIG. 3D). Thus RSV DCs are the most potent tolerogenic DCs as measured by suppression of alloreactive responses.

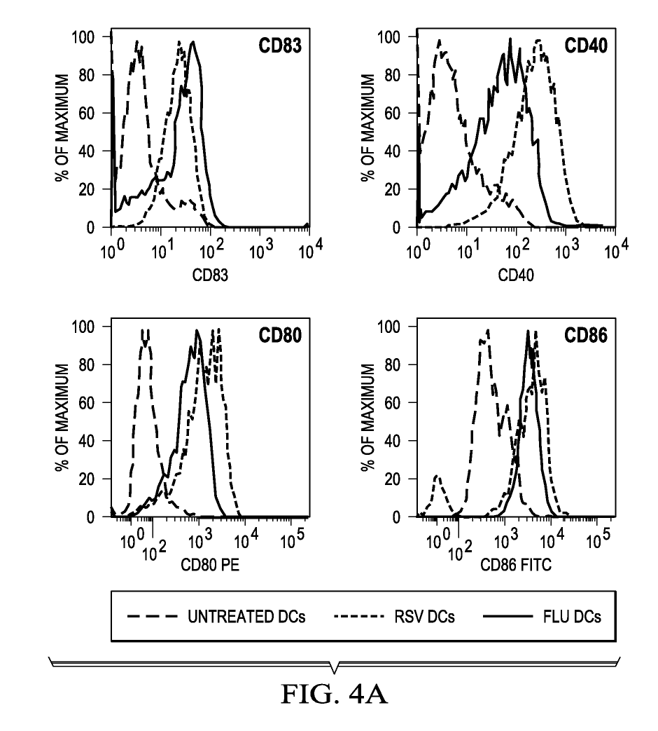

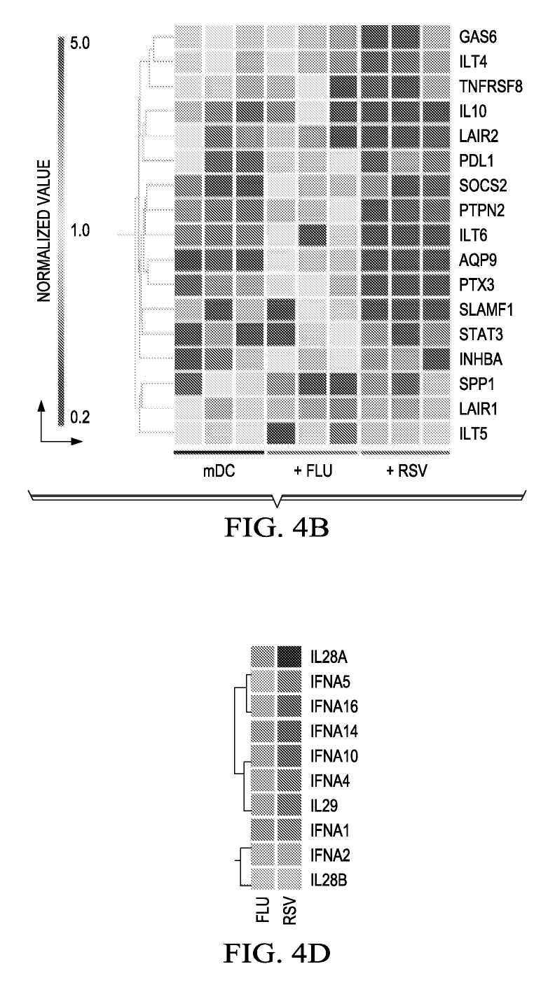

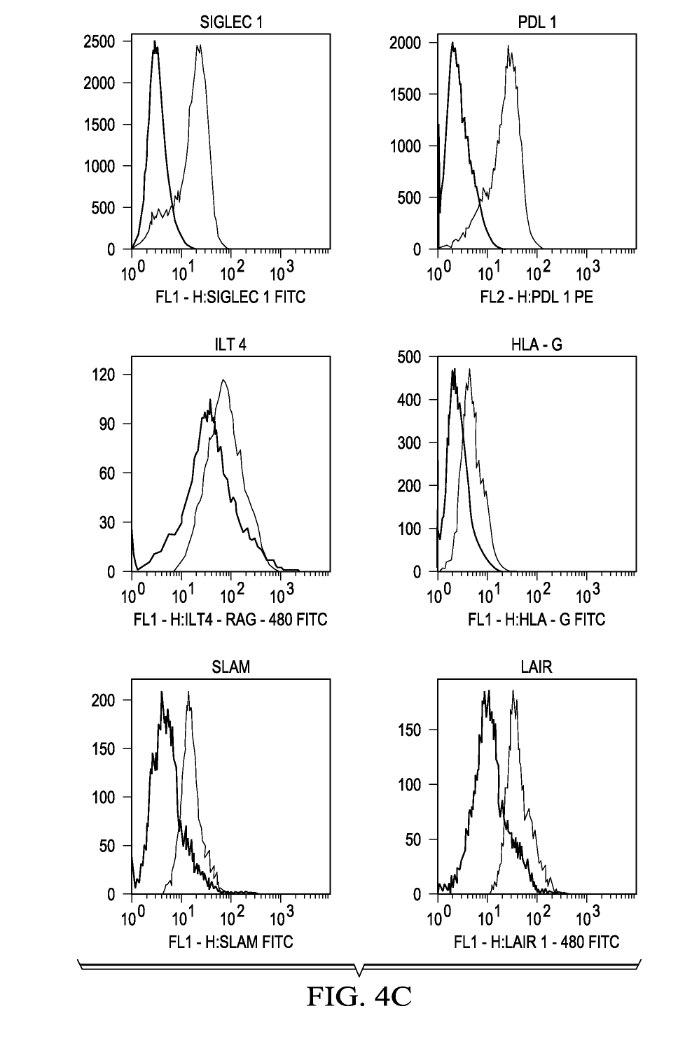

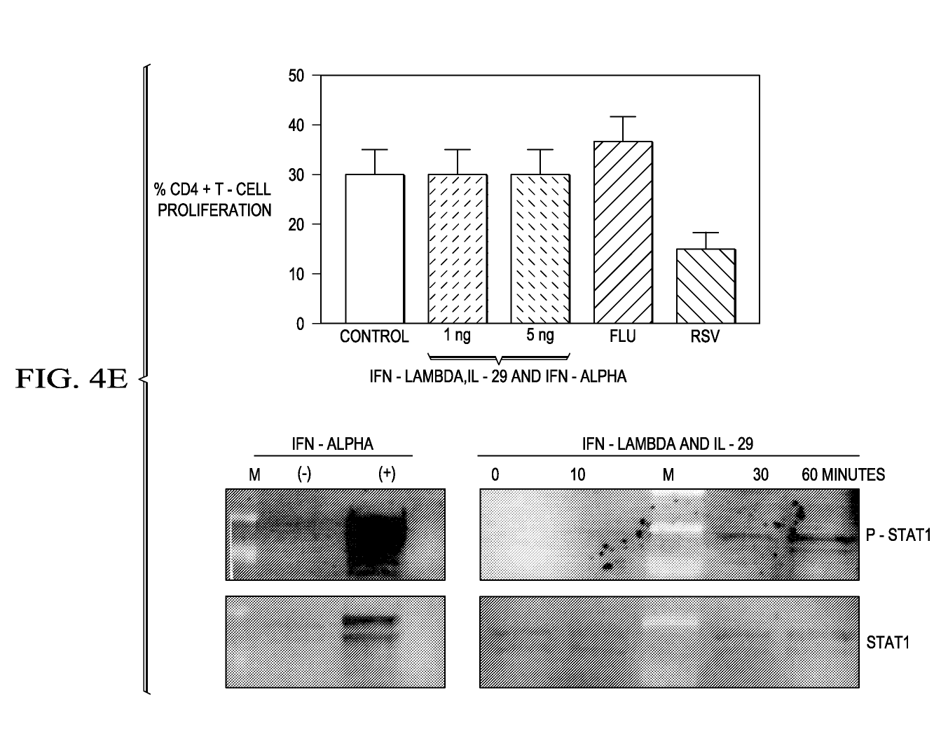

RSV DCs display a unique phenotype. Earlier studies described tolerogenic DCs as expressing low levels of the costimulatory molecules CD80 and CD86^{12,22,23,25}. In contrast, RSV-DCs expressed high levels of CD80 and CD86 (FIG. 4A). The levels of CD40 were consistently higher on RSV-DCs than Flu-DCs whereas the level of CD83 was higher on Flu-DCs (FIG. 4A). These results indicate that the selective inhibition of mDC function observed after RSV exposure is not due to a block in their ability to provide costimulation. To further understand the effect of viral exposure on DCs, we analyzed the RNA transcript profiles of mDCs exposed to either RSV or Flu for 16 hours (FIG. 4B). A striking feature of the RSV specific gene expression profiling was the upregulation of molecules associated with inhibitory function. These molecules fell into two main classes, ITIM containing inhibitory receptors and down stream transducing molecules. The inhibitory class-I immunoreceptors ILT4, ILT5 and ILT6, have been associated with tolerogenic function in DCs^{26,27}. The ITIM containing receptors, LAIR1 and LAIR2 inhibit DC differentiation²⁸. The ITIM containing receptor SLAMF1 is upregulated in tolerogenic DCs and IL-10 treated monocytes^{29,30}. SOCS2,for suppressor of cytokine signaling, is a negative regulator of DC inflammatory cytokine production³¹. STAT3 is a mediator of IL-10 receptor signaling, whose activation is necessary for many of its immunosuppressive properties³². Similarly, the protein tyrosine phosphatase PTPN2 is a critical negative regulator of inflammatory signaling³³. The expression of some ITIM containing inhibitory receptors and ligands was subsequently confirmed on the surface of mDCs 24 hours after RSV exposure (FIG. 4C). RSV-DCs demonstrated a significant increased expression of surface PD-L1. T-cell recognition of PD-L1 inhibits IL-2 production and mediates CD4+ T-cell tolerance to self antigens^{34,35}. Furthermore, engagement of PD-L1 on dendritic cells has been show to directly induce DC IL-10 production^{36,37}. Interestingly, RSV exposure induced the expression of both ILT-4 and its high affinity ligand HLA-G. Each of these molecules is IL-10 inducible and associated with tolerogenic dendritic cell function^{26,27,38,39}.

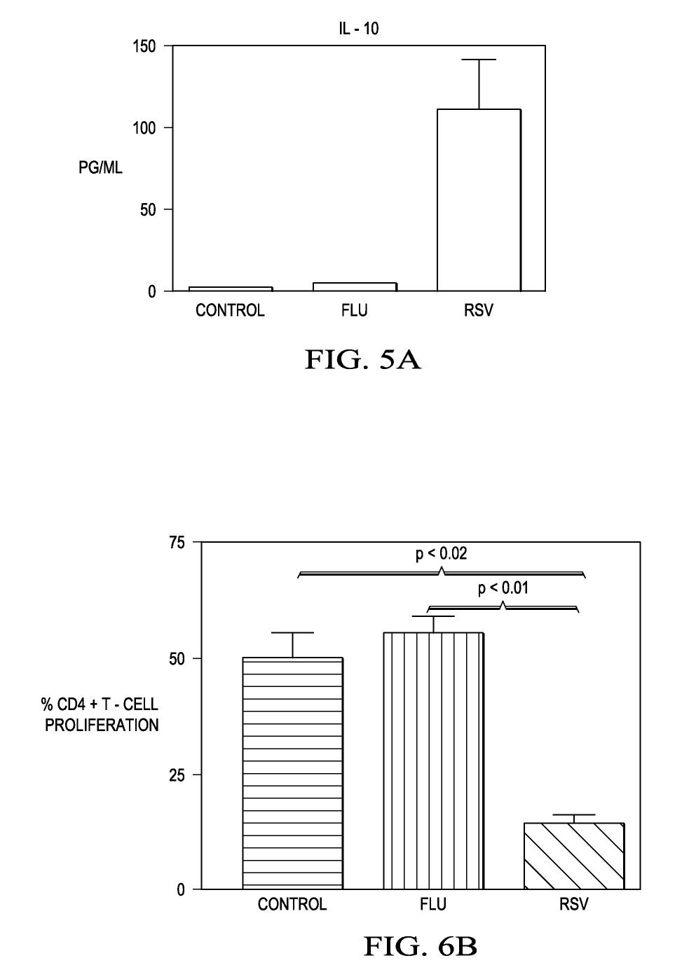

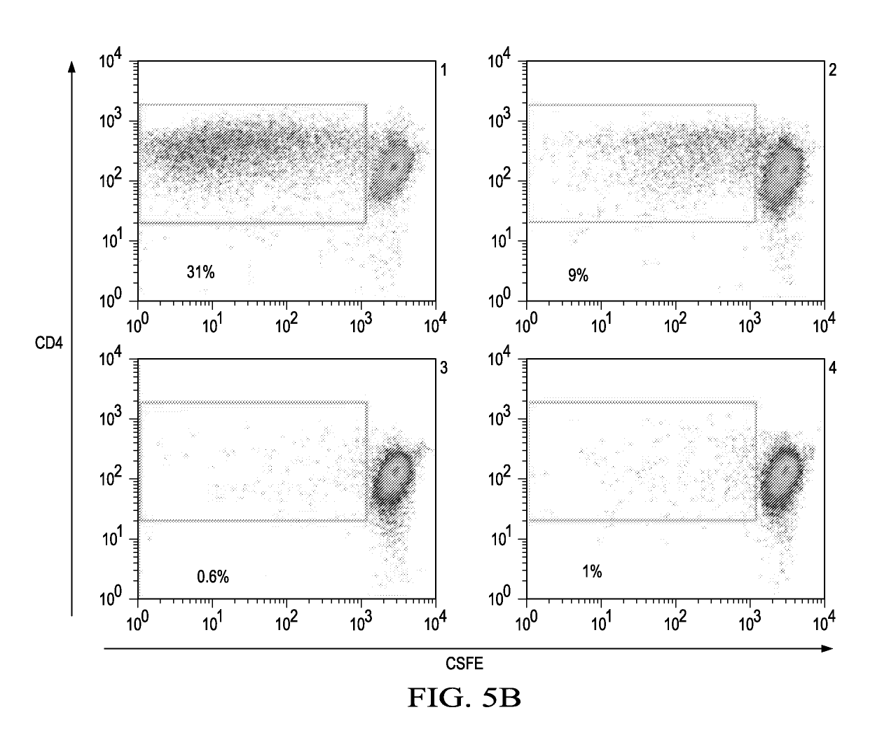

Autocrine IL-10 is required for tolerogenic conversion. Exposure of GMCSF/IL-4 monocyte derived DCs to RSV in vitro has been reported to induce a number of potentially suppressive factors, including IFN-alpha, IFN-lambda and IL-29⁴⁰. However, these factors were not expressed in primary human mDCs in response to RSV exposure (FIG. 4D). Rather, Flu was a potent inducer of IFN-alpha, IFN-lambda and IL-29 and Flu-exposed DCs retained powerful allo stimulatory capability (FIG. 4D). Furthermore the addition of these factors to allo proliferation assays failed to suppress CD4+ T-cell expansion (FIG. 4E). The ability of RSV, but not Flu to induce IL-10 transcription (FIG. 4B) and secretion (FIG. 5A) led us to investigate whether it was involved in the tolerogenic conversion of mDC. Blocking IL-10 receptor signaling (with antibodies to IL-10 and IL-10 receptor) during the allo proliferation assay did not permit the proliferation of allogeneic CD4+ T-cells (FIG. 5B panel 4). However, blocking IL-10 receptor signaling during viral exposure can partially recover the allo stimulatory capacity of RSV-exposed mDCs (FIG. 5B panel 2). These results indicate that autocrine IL-10 production during RSV mediated maturation of the mDC plays a critical role in tolerogenic conversion. Furthermore these results indicate that once the mDC has been exposed to the virus, IL-10 is dispensable for the RSV-DC allo-inhibitory effect.

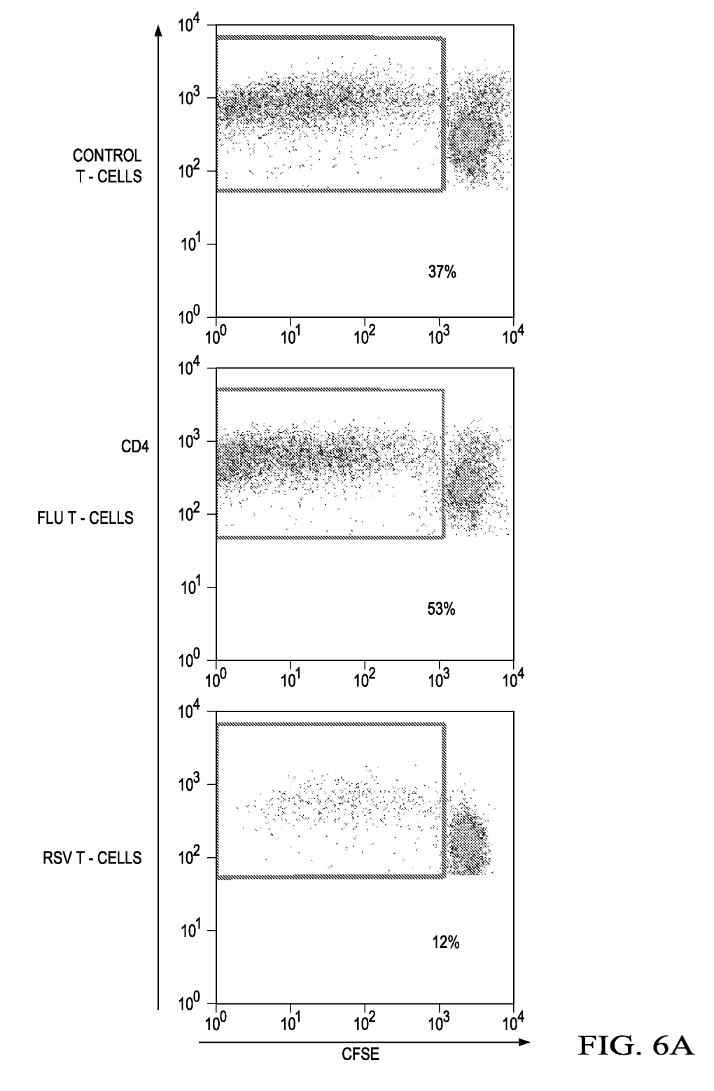

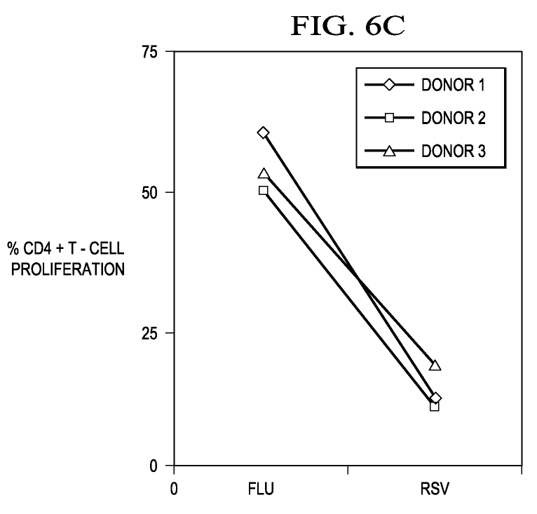

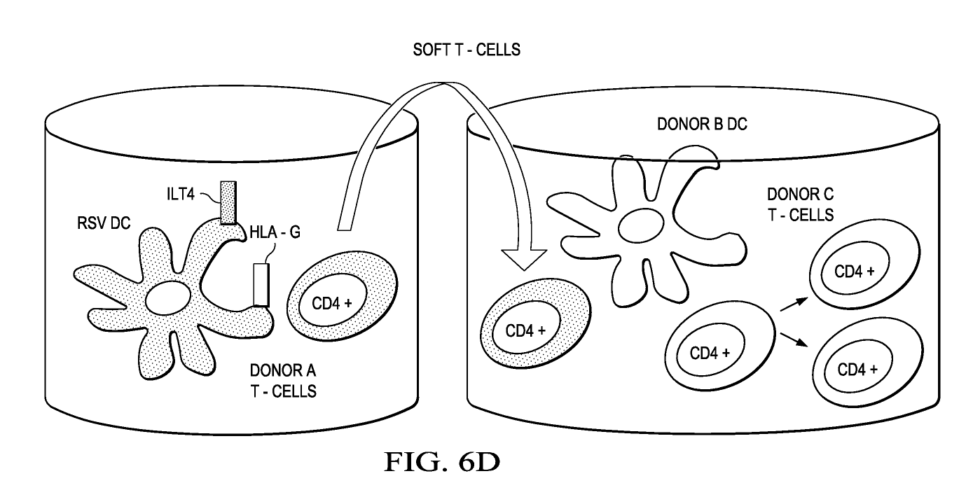

RSV exposed DCs induce Tregs. The ability of a minority of RSV-exposed mDCs to inhibit allogeneic CD4 + T-cell proliferation triggered by non-exposed mDCs, led us to consider that they might induce the differentiation of regulatory T-cells. CFSE labeled allogeneic CD4+ T-cell were therefore cultured with either unexposed, flu or RSV exposed mDCs. As regulatory T-cells have been reported to have limited proliferative capacity, allogeneic CD4+ T-cell populations which divided only once or twice, were isolated by cell sorting following 5 days of DC coculture^{41,42}. Next, 1,500 CD4+ T-cells sorted from unexposed, flu or RSV exposed mDCs cocultures were thus added to MLR consisting of 1,250 unexposed mDCs and 500,000 labeled CD4+ T-cells. As shown in FIG. 6, T-cells derived from RSV exposed mDCs could themselves inhibit unexposed mDC induced alloreaction. In contrast T-cells derived from unexposed or Flu exposed DCs showed no inhibitory effect (FIG. 6). Thus RSV exposed DCs are potent inducers of regulatory T-cells.

These studies on the interaction of RSV with DCs yield two main conclusions which might explain why the adaptive immune response to RSV in humans is inefficient and repeat infections occur throughout an individual's lifetime. First, RSV infection blocks APC function during natural infection. Acute RSV infection results in a severe defect in allo antigen presenting capability of blood APCs. mDCs isolated from the site of infection are likewise unable to mount an alloproliferative response. Our in vitro studies demonstrate that the immune suppression observed in patients may be the result of a direct effect of RSV on DCs.

The second main conclusion derived from these studies is that RSV induces DCs to develop powerful tolerogenic function. Indeed, remarkably few RSV-DCs are capable of inhibiting alloproliferative responses in trans. In our hands this suppressive function is more potent than previously described pharmacologically generated tolerogenic dendritic cells. The ability of RSV-exposed mDCs to induce regulatory T-cells points to a role for these cells in propagating this inhibitory signal. Indeed, as with RSV-DCs themselves, very few (1500) T-cells which have been exposed to RSV-DCs can inhibit alloreactions performed with 100,000 T-cells.

Though a wide variety of inhibitory receptors and ligands are upregulated by RSV mechanism of tolerogenic DC suppression remains undefined. The necessity for cell to cell contact, and inhibitory ability of fixed cells, precludes a direct role of soluble inhibitory factors, such as IL-10 or IFN-lambda, in this suppression. RSV mediated induction of tolerogenic DCs may explain the inefficient generation of RSV specific immunity. RSV induces tolerogenic DCs by skewing DC maturation through a mechanism which requires autocrine IL-10. These DCs are then capable of driving the differentiation of regulatory CD4+ T-cells. This effective mechanism of immune subversion has implications not only on RSV vaccine design but also in the treatment of hyper-immune disorders such as auto-immune disease and organ transplant.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Henderson, F. W., Collier, A. M., Clyde, W. A., Jr. & Denny, F. W. Respiratory-syncytial-virus infections, reinfections and immunity. A prospective, longitudinal study in young children. N. Engl. J. Med. 300, 530-534 (1979).
2. Zambon, M. C., Stockton, J. D., Clewley, J. P. & Fleming, D. M. Contribution of influenza and respiratory syncytial virus to community cases of influenza-like illness: an observational study. Lancet 358, 1410-1416 (2001).
3. Hall, C. B., Walsh, E. E., Long, C. E. & Schnabel, K. C. Immunity to and frequency of reinfection with respiratory syncytial virus. J. Infect. Dis. 163, 693-698 (1991).
4. Hall, C. B., Long, C. E. & Schnabel, K. C. Respiratory syncytial virus infections in previously healthy working adults. Clin. Infect. Dis. 33, 792-796 (2001).
5. Chin, J., Magoffin, R. L., Shearer, L. A., Schieble, J. H. & Lennette, E. H. Field evaluation of a respiratory syncytial virus vaccine and a trivalent parainfluenza virus vaccine in a pediatric population. Am. J. Epidemiol. 89, 449-463 (1969).
6. Fulginiti, V. A. et al. Respiratory virus immunization. I. A field trial of two inactivated respiratory virus vaccines; an aqueous trivalent parainfluenza virus vaccine and an alum-precipitated respiratory syncytial virus vaccine. Am. J. Epidemiol. 89, 435-448 (1969).
7. Kapikian, A. Z., Mitchell, R. H., Chanock, R. M., Shvedoff, R. A. & Stewart, C. E. An epidemiologic study of altered clinical reactivity to respiratory syncytial (RS) virus infection in children previously vaccinated with an inactivated RS virus vaccine. Am. J. Epidemiol. 89, 405-421 (1969).
8. Banchereau, J. & Steinman, R. M. Dendritic cells and the control of immunity. Nature 392, 245-252 (1998).
9. Palucka, K. & Banchereau, J. How dendritic cells and microbes interact to elicit or subvert protective immune responses. Curr. Opin. Immunol. 14, 420-431 (2002).
10. Tortorella, D., Gewurz, B. E., Furman, M. H., Schust, D. J. & Ploegh, H. L. Viral subversion of the immune system. Annu. Rev. Immunol. 18, 861-926 (2000).
11. Steinman, R. M., Hawiger, D. & Nussenzweig, M. C. Tolerogenic dendritic cells. Annu Rev. Immunol. 21, 685-711 (2003).
12. Roncarolo, M. G., Levings, M. K. & Traversari, C. Differentiation of T regulatory cells by immature dendritic cells. J. Exp. Med. 193, F5-F9 (2001).
13. Steinman, R. M. The control of immunity and tolerance by dendritic cell. Pathol Biol (Paris) 51, 59-60 (2003).
14. Bergwelt-Baildon, M. S. et al. CD25 and indoleamine 2,3-dioxygenase are up-regulated by prostaglandin E2 and expressed by tumor-associated dendritic cells in vivo: additional mechanisms of T-cell inhibition. Blood 108, 228-237 (2006).
15. Min, S. Y. et al. Antigen-induced, tolerogenic CD11c+, CD11b+ dendritic cells are abundant in Peyer's patches during the induction of oral tolerance to type II collagen and suppress experimental collagen-induced arthritis. Arthritis Rheum. 54, 887-898 (2006).
16. Fujita, S. et al. Regulatory dendritic cells act as regulators of acute lethal systemic inflammatory response. Blood 107, 3656-3664 (2006).
17. Moser, M. Dendritic cells in immunity and tolerance-do they display opposite functions? Immunity 19, 5-8 (2003).
18. Probst, H. C., McCoy, K., Okazaki, T., Honjo, T. & Van Den, B. M. Resting dendritic cells induce peripheral CD8+ T cell tolerance through PD-1 and CTLA-4. Nat. Immunol. 6, 280-286 (2005).
19. Gill, M. A. et al. Mobilization of plasmacytoid and myeloid dendritic cells to mucosal sites in children with respiratory syncytial virus and other viral respiratory infections. J. Infect. Dis. 191, 1105-1115 (2005).
20. Jones, A., Morton, I., Hobson, L., Evans, G. S. & Everard, M. L. Differentiation and immune function of human dendritic cells following infection by respiratory syncytial virus. Clin. Exp. Immunol. 143, 513-522 (2006).
21. de Graaff, P. M. et al. Respiratory syncytial virus infection of monocyte-derived dendritic cells decreases their capacity to activate CD4 T cells. J. Immunol. 175, 5904-5911 (2005).
22. Woltman, A. M. et al. The effect of calcineurin inhibitors and corticosteroids on the differentiation of human dendritic cells. Eur. J. Immunol. 30, 1807-1812 (2000).
23. Xia, C. Q., Peng, R., Beato, F. & Clare-Salzler, M. J. Dexamethasone induces IL-10-producing monocyte-derived dendritic cells with durable immaturity. Scand. J. Immunol. 62, 45-54 (2005).
24. Steinbrink, K., Graulich, E., Kubsch, S., Knop, J. & Enk, A. H. CD4(+) and CD8(+) anergic T cells induced by interleukin-10-treated human dendritic cells display antigen-specific suppressor activity. Blood 99, 2468-2476 (2002).
25. Pedersen, A. E., Gad, M., Walter, M. R. & Claesson, M. H. Induction of regulatory dendritic cells by dexamethasone and 1alpha,25-Dihydroxyvitamin D(3). Immunol. Lett. 91, 63-69 (2004).
26. Chang, C. C. et al. Tolerization of dendritic cells by T(S) cells: the crucial role of inhibitory receptors ILT3 and ILT4. Nat. Immunol. 3, 237-243 (2002).
27. Manavalan, J. S. et al. High expression of ILT3 and ILT4 is a general feature of tolerogenic dendritic cells. Transpl. Immunol. 11, 245-258 (2003).
28. Poggi, A., Tomasello, E., Ferrero, E., Zocchi, M. R. & Moretta, L. p40/LAIR-1 regulates the differentiation of peripheral blood precursors to dendritic cells induced by granulocyte-monocyte colony-stimulating factor. Eur. J. Immunol. 28, 2086-2091 (1998).
29. Velten, F. W., Duperrier, K., Bohlender, J., Metharom, P. & Goerdt, S. A gene signature of inhibitory MHC receptors identifies a BDCA3(+) subset of IL-10-induced dendritic cells with reduced allostimulatory capacity in vitro. Eur. J. Immunol. 34, 2800-2811 (2004).
30. Jung, M. et al. Expression profiling of IL-10-regulated genes in human monocytes and peripheral blood mononuclear cells from psoriatic patients during IL-10 therapy. Eur. J. Immunol. 34, 481-493 (2004).
31. Machado, F. S. et al. Anti-inflammatory actions of lipoxin A4 and aspirin-triggered lipoxin are SOCS-2 dependent. Nat. Med. 12, 330-334 (2006).
32. Kortylewski, M. et al Inhibiting Stat3 signaling in the hematopoietic system elicits multicomponent antitumor immunity. Nat. Med. 11, 1314-1321 (2005).
33. van Vliet, C. et al. Selective regulation of tumor necrosis factor-induced Erk signaling by Src family kinases and the T cell protein tyrosine phosphatase. Nat. Immunol. 6, 253-260 (2005).
34. Keir, M. E. et al. Tissue expression of PD-L1 mediates peripheral T cell tolerance. J. Exp. Med. 203, 883-895 (2006).
35. Sharpe, A. H., Wherry, E. J., Ahmed, R. & Freeman, G. J. The function of programmed cell death 1 and its ligands in regulating autoimmunity and infection. Nat. Immunol. 8, 239-245 (2007).
36. Kuipers, H. et al. Contribution of the PD-1 ligands/PD-1 signaling pathway to dendritic cell-mediated CD4+ T cell activation. Eur. J. Immunol. 36, 2472-2482 (2006).
37. Van Keulen, V. P. et al. Immunomodulation using the recombinant monoclonal human B7-DC cross-linking antibody rHIgM12. Clin. Exp. Immunol. 143, 314-321 (2006).
38. Moreau, P. et al. IL-10 selectively induces HLA-G expression in human trophoblasts and monocytes. Int. Immunol. 11, 803-811 (1999).
39. Spencer, J. V. et al. Potent immunosuppressive activities of cytomegalovirus-encoded interleukin-10. J. Virol. 76, 1285-1292 (2002).
40. Chi, B. et al. Alpha and lambda interferon together mediate suppression of CD4 T cells induced by respiratory syncytial virus. J. Virol. 80, 5032-5040 (2006).
41. Malek, T. R. & Bayer, A. L. Tolerance, not immunity, crucially depends on IL-2. Nat. Rev. Immunol. 4, 665-674 (2004).
42. Lohr, J., Knoechel, B. & Abbas, A. K. Regulatory T cells in the periphery. Immunol. Rev. 212, 149-162 (2006).

What is claimed is:

1. A method for inducing immune tolerance using antigen presenting cells comprising;
infecting isolated antigen presenting cells with an effective amount of respiratory syncytial virus (RSV) or portions thereof sufficient to infect the antigen presenting cells; and
contacting CD4⁺, CD8⁺ or both CD4⁺ T cells and CD8⁺ T cells with the RSV-infected antigen presenting cells, wherein the CD4⁺, CD8⁺ or both CD4 and CD8+ T cells are rendered tolerogenic as measured in vitro by a mixed leukocyte reaction.

2. The method of claim 1, wherein the RSV-infected antigen presenting cells are peripheral blood mononuclear cells, immature dendritic cells, mature dendritic cells or Langerhans cells.

3. The method of claim 1, wherein the RSV-infected antigen presenting cells are tolerogenic at a ratio of 1:1 to 1:100 tolerogenic antigen presenting cells to T cells.

4. The method of claim 1, wherein the RSV-infected cells are fixed prior to contacting with the T cells.

5. The method of claim 1, wherein the RSV-infected antigen presenting cells are CD80^{high}, CD86^{high}, CD40^{high} and CD83^{low}.

6. The method of claim 1, wherein the RSV-infected antigen presenting cells are CD80^{high}, CD86^{high}, CD40^{high} and CD83^{low}, when compared to Flu infected antigen presenting cells.

7. The method of claim 1, wherein the RSV-infected antigen presenting cells induce the proliferation of regulatory T-cells.

8. The method of claim 1, wherein the RSV-infected antigen presenting cells secrete IL-10 and have increased expression over untreated antigen presenting cells of SIGLEC-1, PDL-1, ILT-4, HLA-G, SLAM and LAIR.

9. The method of claim 1, wherein the RSV-infected antigen presenting cells have an increase in gene expression, when compared to untreated antigen presenting cells, of IL-10, LAIR2, SOCS2, PTPN2, ILT-6, AQP9, PTX3 and SLAMF1.

10. A method for making tolerizing dendritic cells comprising;
infecting dendritic cells with effective amount of respiratory syncytial virus to develop IL-10 dependent tolerogenic immune function, wherein respiratory syncytial virus increased the dendritic cells' ability to tolerize allogeneic CD4+ T-cells, cause suppressor T-cell proliferation, secrete IL-10 and express inhibitory molecules PDL-1, ILT-4 and HLA-G and wherein the infecting dendritic cells are CD80^{high}, CD86^{high}, CD40^{high} and CD83^{low}.

11. The method of claim 10, wherein the inhibition of dendritic cells' ability to activate allogeneic CD4₊ T-cell requires cell-to-cell contact between dendritic cells.

12. A method for suppressing antiviral immunity of dendritic cells in a subject comprising;
infecting isolated dendritic cells with effective amount of respiratory syncytial virus to develop IL-10 dependent tolerogenic immune function, wherein respiratory syncytial virus inhibit the dendritic cells' ability to activate allogeneic CD4+ T-cells, induce naïve T-cell regulatory response, secrete IL-10 and express inhibitory molecules PDL-1, IKT-4, and HLA-G when reintroduced into a patient.

13. The method of claim 12, wherein the inhibition of dendritic cells' ability to activate allogeneic CD4+ T-cell requires cell-to-cell contact between dendritic cells.

14. A tolerogenic dendritic cell comprising an isolated dendritic cells that is CD80^{high}, CD86^{high}, CD40^{high} and CD83^{low}.

15. A tolerogenic dendritic cell made by the method of infecting peripheral blood mononuclear cells with an effective amount of a respiratory syncytial virus or portions thereof sufficient to rendered CD4⁺, CD8⁺ or both CD4⁺ T cells and CD8⁺ T cells tolerogenic as measured in vitro by a mixed leukocyte reaction and wherein the dendritic cells that is CD80^{high}, CD86^{high}, CD40^{high} and CD83^{low}.

16. A method of promoting tolerogenic T cell-mediated immune responses by contacting the T cells with a dendritic cell that has been infected with an amount of a RSV or portion thereof sufficient to trigger the surface expression of at least one of CD80^{high}, CD86^{high}, CD40^{high} and CD83^{low}.

17. A method of inducing anergic T helper cells which comprises:
incubating isolated antigen presenting cells (APC) with an amount of RSV sufficient to infect the antigen presenting cell and trigger the surface expression of at least one of the following cell surface markers CD80^{high}, CD86^{high}, CD40^{high} and CD83^{low}; and
contacting the RSV-infected antigen presenting cells with T cells under conditions that tolerize the T cells as measured in vitro in a mixed lymphocyte reaction.

18. A method of producing an isolated tolerogenic dendritic cell comprising:
incubating the isolated dendritic cell with an amount of respiratory syncytial virus sufficient to infect the dendritic cell under conditions that trigger the cell surface expression the following cell surface CD80^{high}, CD86^{high}, CD40^{high} and CD83^{low}.

19. A kit for enhancing tolerogenicity in a mammalian host comprising isolated tolerogenic dendritic cells previously infected with RSV and having the following cell surface CD80^{high}, CD86^{high}, CD40^{high} and CD83^{low}.

20. A method of generating a tolerogenic antigen presenting cell (APC) comprising:
infecting the APC with an amount of respiratory syncytial virus sufficient to infect the dendritic cell; and
causing the following cell surface marker expression CD80^{high}, CD86^{high}, CD40^{high} and CD83^{low} thereby generating a tolerogenic antigen presenting cell (APC).

21. A method for treating an autoimmune disease in a mammalian subject, comprising administering to the mammalian subject tolerogenic antigen presenting cell (APC), wherein the tolerogenic dendritic cells previously infected with RSV and having the following cell surface CD80^{high}, CD86^{high}, CD40^{high} and CD83^{low}, and the cells are administered in an amount effective to reduce or eliminate the autoimmune disease or to prevent its occurrence or recurrence.

22. The method of claim 21, wherein the autoimmune disease is insulin-dependent diabetes mellitus, multiple sclerosis, autoimmune encephalomyelitis, rheumatoid arthritis, autoimmune arthritis, myasthenia gravis, thyroiditis, uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, psoriasis sympathetic ophthalmia, phacogenic uveitis, autoimmune haemolytic anaemia, idiopathic leucopenia, primary biliary cirrhosis, active chronic hepatitis, cryptogenic cirrhosis, ulcerative colitis, Sjogren's syndrome, scleroderma, Wegener's granulomatosis, poly/dermatomyositis, discoid lupus erythematosus or systemic lupus erythematosus.

23. A method for modulating the immune response to an antigen, comprising administering to a patient in need of such treatment an isolated tolerizing antigen-presenting cell for a time and under conditions sufficient to modulate the immune response, wherein the antigen-specific antigen-presenting cell is produced by contacting the antigen-presenting cell with RSV for a time and under conditions sufficient for the antigen-presenting cell to become a tolerizing to T cells, wherein the tolerizing antigen-presenting cell is characterized by expressing the following cell surface markers CD80^{high}, CD86^{high}, CD40^{high} and CD83^{low}, and wherein the tolerizing antigen presenting cell is tolerogenic at a ratio of 1:5 to 1:100 tolerizing antigen presenting cells to T cells.
